(19) United States
(12) Patent Application Publication
(10) Pub. No.: US 2024/0399107 A1
(43) Pub. Date: Dec. 5, 2024

(54) DIRECTIONAL TIPPED CATHETERS

(71) Applicant: Brian B. MARTIN, Santa Cruz, CA (US)

(72) Inventor: Brian B. MARTIN, Santa Cruz, CA (US)

(21) Appl. No.: 18/679,959

(22) Filed: May 31, 2024

Related U.S. Application Data
(60) Provisional application No. 63/505,399, filed on May 31, 2023.

(51) Int. Cl. A61M 25/00

(52) U.S. Cl. CPC A61M 25/0068; A61M 25/0074; A61M 25/008

(57) ABSTRACT

Catheters and medical devices having tip member configured to bend the tip in a direction upon deformation of structures on the tip member.

### CROSS-REFERENCE TO RELATED APPLICATIONS

This application is a non-provisional of U.S. Provisional application No. 63/505,399 filed May 31, 2023, the entirety of which is incorporated by reference.

### FIELD OF THE INVENTION

Catheters having improved navigational tips.

### BACKGROUND OF THE INVENTION

Medical catheters allow physicians to apply a variety of different therapies within the body of a patient. Many catheters access remote regions of the human body for delivering diagnostic or therapeutic tools and/or agents to those sites. Alternatively, the catheter can comprise a shaft or support for a therapeutic working end (e.g., balloon, filter retriever, electrode, etc.). Some catheters, including but not limited to catheters for neurovascular use, are intended to be advanced from a main artery (e.g., a femoral or radial artery) through tortuous anatomy into a small cerebral vessel. As such, the catheter must be configured with varying structural traits due to the varying regions of the anatomy through which the catheter passes. Many times, the vascular pathways wind back upon themselves in a multi-looped path making it difficult for catheter design to meet the requirements demanded by the tortuous anatomy. For example, catheters must be fairly stiff at their proximal end so as to allow the pushing and manipulation of the catheter as it progresses through the body, and yet must be sufficiently flexible at the distal end to allow passage of the catheter tip through the loops and smaller blood vessels. Regardless, the catheter does not cause significant trauma to the blood vessel or to the surrounding tissue.

Many conventional catheters rely on a soft distal tip to minimize the risk of causing trauma to a vessel, freeing plaque from a vessel wall, puncturing a vessel, or creating embolisms in the bloodstream. Conventional catheters 50, as shown in FIG. 1A include a hub 52 coupled to a catheter body 54 with a soft tip 56 that has a conventional flat or square tip 58. In those cases where the catheter 52 is advanced within vessel 10 and encounters a branching vessel 12, the flat conventional tip 58 engages the back wall of the branching vessel 12 and does not deflect to either side, as shown in FIG. 1B.

In many cases, catheters must be advanced through tortuous anatomy with a decreasing vessel diameter. In such cases, the diameter of the catheter is significantly reduced, and navigation is difficult in the absence of a catheter tip that can bend to assist in navigation.

Therefore, there remains a need for a catheter having improved features to improve navigation of the catheter through tortuous anatomy.

### SUMMARY OF THE INVENTION

The present disclosure includes catheters and similar medical devices, a tip structure located at a distal end of the body of the device where the tip structure allows for preferential bending when advanced against a structure in the body. In one example, such a catheter construction can include a catheter body having a catheter lumen extending therethrough; and a tip structure located at a distal end of the catheter body, where an end of the tip structure located opposite to the tubular member comprises an undulating shape forming a plurality of scalloped members, where each scalloped member of the plurality of scalloped members is configured to flex individually from an adjacent scalloped member, such that when advanced against a vessel wall, flexing of one or more of the plurality of scalloped members causes articulation of the tip structure.

Variations of the catheter construction include at least one material strip extending in the tip member, where the material strip comprises a first durometer, where the first durometer is different than a second durometer of the remainder of the tip structure. The first durometer can be greater than or less than the second durometer.

Variations of the device include a material strip that extends in alignment with an axis of the tip structure. Alternatively, the at least one material strip can extend spirally about the tip structure.

Likewise, variations of the catheter structure can include a plurality of scalloped members that extend parallel to an axis of the tip structure. Alternatively, the plurality of scalloped members extends at an angle to an axis of the tip structure such that they are twisted at the far end of the tip structure.

One or more of the scalloped members can be configured to deflect towards an axis of the tip member or away from an axis of the tip member.

In a further variation, at least one of the plurality of scalloped members can comprise a neck area having a reduced surface area relative to the remainder of the at least one scalloped member.

In another variation, the present disclosure includes a catheter construction for use in a vessel, the catheter construction comprising: a catheter body having a catheter lumen extending therethrough; and a plurality of scalloped members located at a distal end of the catheter body and formed from an undulating shape pattern such that each scalloped member of the plurality of scalloped members is configured to flex individually from an adjacent scalloped member, such that when advanced against a vessel wall, flexing of one or more of the plurality of scalloped members causes articulation of the tip structure.

The devices of the present disclosure allow for a considerable number of combinations and permutations of different variations of catheters as well as a combination of aspects of those structures as well. It is contemplated that any of the requirements and elements described herein can be combined with any independent claim where the requirements of the independent claims would not contradict the various elements.

### BRIEF DESCRIPTION OF THE FIGURES

FIG. 1A illustrates a traditional catheter construction with a square or uniform tip.

FIG. 1B illustrates the catheter of FIG. 1A encountering a vessel wall.

FIG. 2A shows a partial sectional view of an improved catheter having a tip structure at the distal end of a catheter body.

FIG. 2B illustrates a perspective view of the irregular or scalloped surface forming the scalloped members.

FIGS. 2C and 2D illustrate an additional variation of an improved catheter tip.

FIGS. 3A and 3B show an example of a catheter with an improved navigational tip navigating through a vessel and a branching vessel.

FIGS. 4A to 4D illustrate a non-exhaustive number of variations of improved navigational tips for use with catheters and similar medical devices.

FIGS. 5A to 5D show additional variations of improved navigational tips.

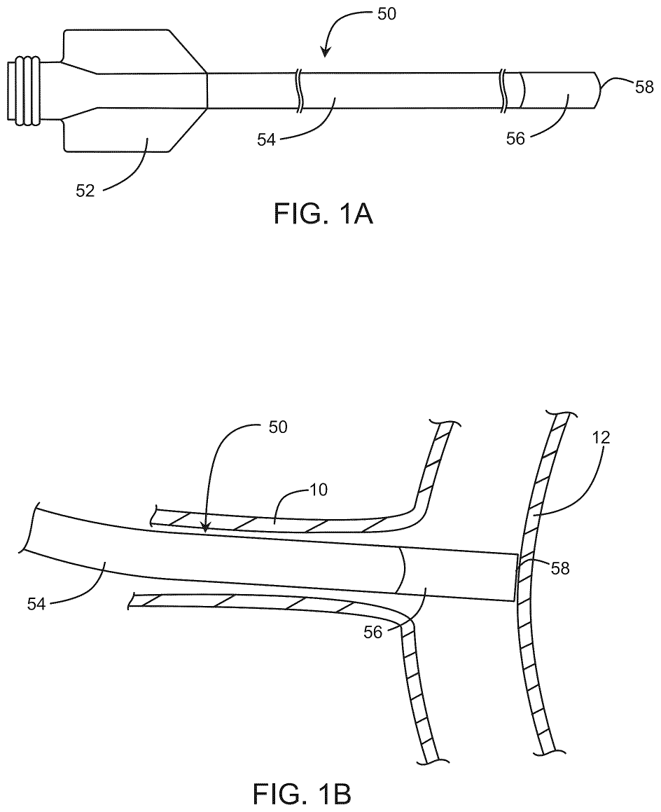

### DETAILED DESCRIPTION

The catheter configuration discussed herein can be used in a variety of devices where different regions are selected for customized properties. The configurations described herein can be incorporated into various medical devices or can be used as catheter shafts. Furthermore, in some variations, the construction features of the present disclosure are not limited to in-dwelling medical devices and can be used for any device requiring improved navigation.

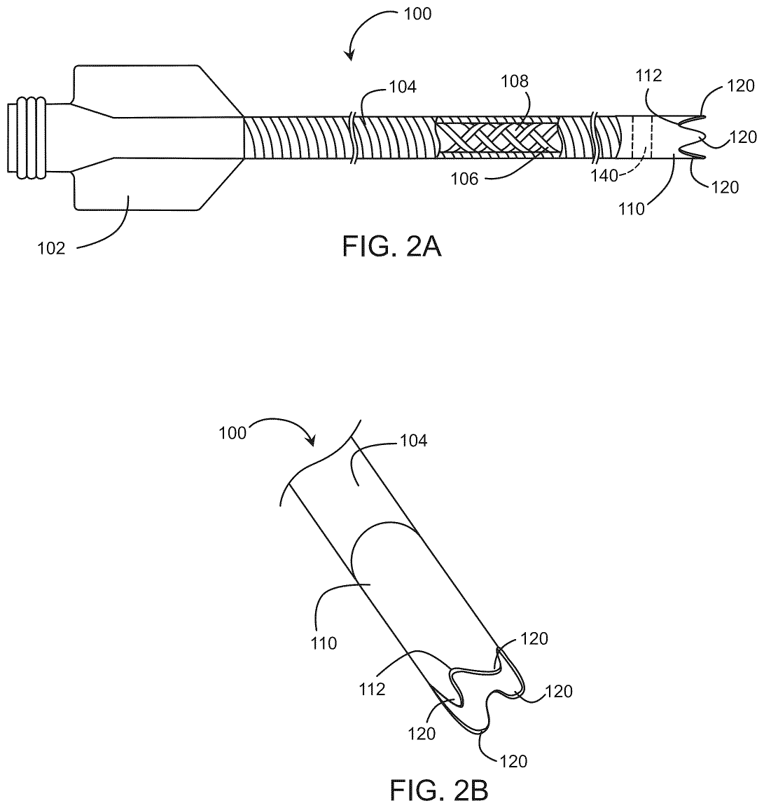

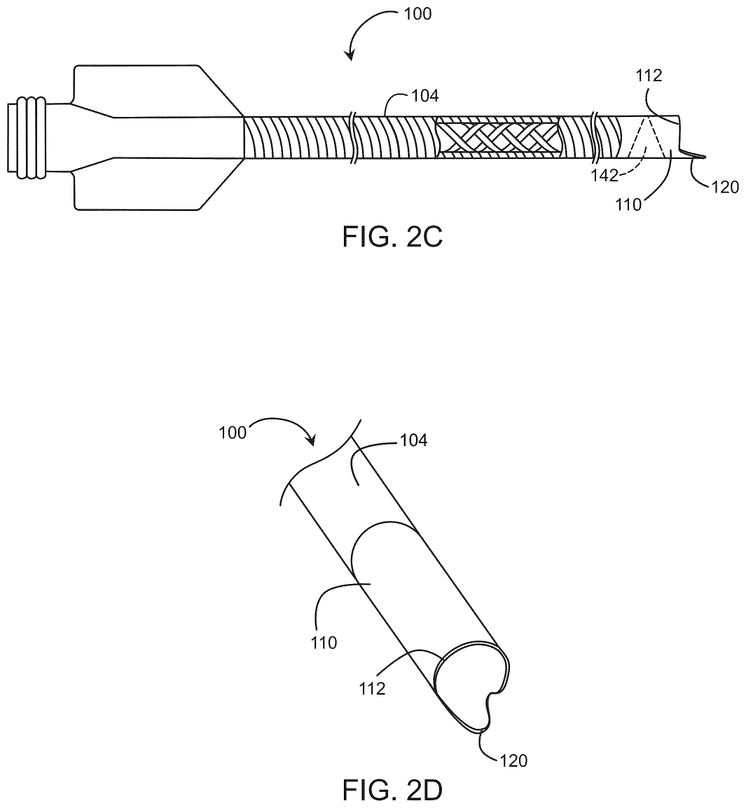

FIG. 2A shows a partial sectional view of an improved catheter 100 having a tip structure 110 at the distal end of a catheter body 104. An end 112 of the tip structure that is opposite to the catheter body 104 includes an undulating or scalloped shape, which forms a plurality of scalloped members 120. Variations of the device 100 can include a tip structure 110 with two or more scalloped members 120. Each scalloped member 120 can flex individually from an adjacent member 120. As discussed below, the ability of the scalloped members 120 to flex allows for articulation of the tip structure 110 and allows improved navigation of the catheter 100. The catheter 100 can further include an optional radiopaque marker 140 located along any portion of the catheter or tip.

FIG. 2A also illustrates the catheter, including a hub 102 at the proximal end of the catheter body 104. The hub shown is for purposes of illustration, and any number of hubs or handle structures can be used. In additional variations of the catheter 100, a hub is not required. The catheter body 104 can comprise any variation of catheter construction, such as an inner braid 106 surrounding an inner liner 108. Moreover, the catheter body 104 can optionally comprise a hybrid polymer construction such as disclosed in U.S. Pat. Nos. 10,898,683, 11,077,285, 11,179,546, and U.S. Provisional patent 63/369,947, each of which is incorporated by reference herein.

FIG. 2B illustrates a perspective view of the irregular or scalloped surface 112 forming the scalloped members 120. While the illustrated variation, the catheter construction 100 shown in FIGS. 2A and 2B illustrates curved scalloped members 120. Additional variations of the device 100 can include triangular or elliptical scalloped members. Additionally, variations of the device 100 include scalloped members 120 having different shapes on a single tip structure 110.

FIG. 2C shows a partial cross section of another variation of a catheter having including a hub 102 at the proximal end of the catheter body 104. In this variation, an end 112 of the tip structure that is opposite to the catheter body 104 includes a single undulating or scalloped shape 120 which flexes allows for articulation of the tip structure 110 and allows improved navigation of the catheter 100. FIG. 2C shows the catheter 100 having an optional radiopaque marker 142 located along any portion of the catheter or tip. In this variation, the marker 142 comprises an irregular shape that allows detection of a location of the scalloped shape when viewed fluoroscopically. FIG. 2D illustrates a perspective view of the irregular or scalloped surface 112 forming a single scalloped members 120.

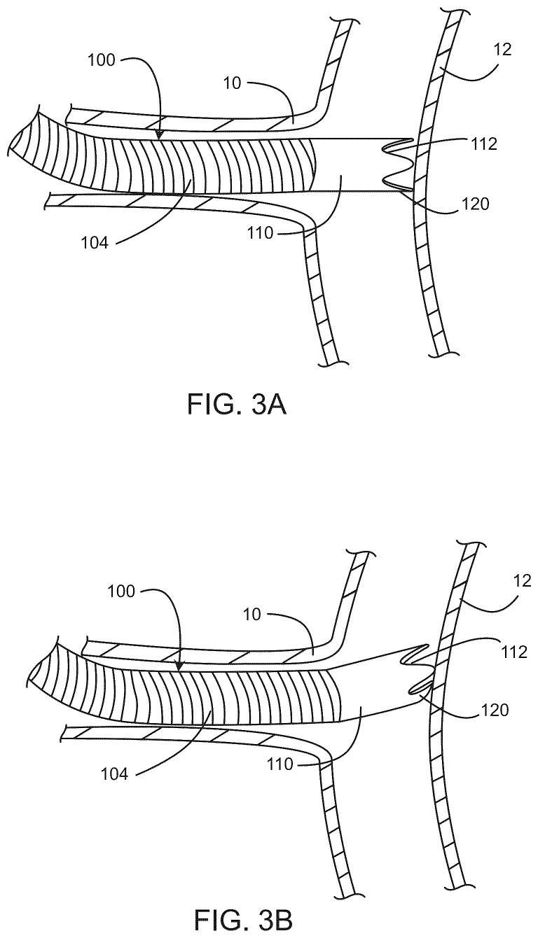

FIGS. 3A and 3B show an example of a catheter 100 with an improved navigational tip 110. FIG. 3A illustrates the catheter 100 advanced through a vessel 10 into a branching vessel 12. As the catheter approaches a far wall of the branching vessel 12, one or more scalloped members 120 engages a wall and deflects, as shown in FIG. 3B. However, because of the non-uniform surface of the end 112 of the tip member 110, the remaining scalloped members do not deflect. The deflection of the affected scalloped members 120 causes articulation of the tip member 110 and catheter body 104, allowing for improved navigation of the catheter 100 through tortuous anatomy. It is noted that FIG. 3B illustrates the tip member 110 deflected upwards. However, in certain variations, the design of the tip member 110 and scalloped members 120 can cause articulation in any direction. Therefore, as compared to a conventional catheter tip, this preferential bending in either direction allows the user to navigate the catheter 100 by withdrawal and advancement of the tip member 110 until the tip member 110 asymmetrically or non-uniformly bends allowing for improved navigation of the catheter 100.

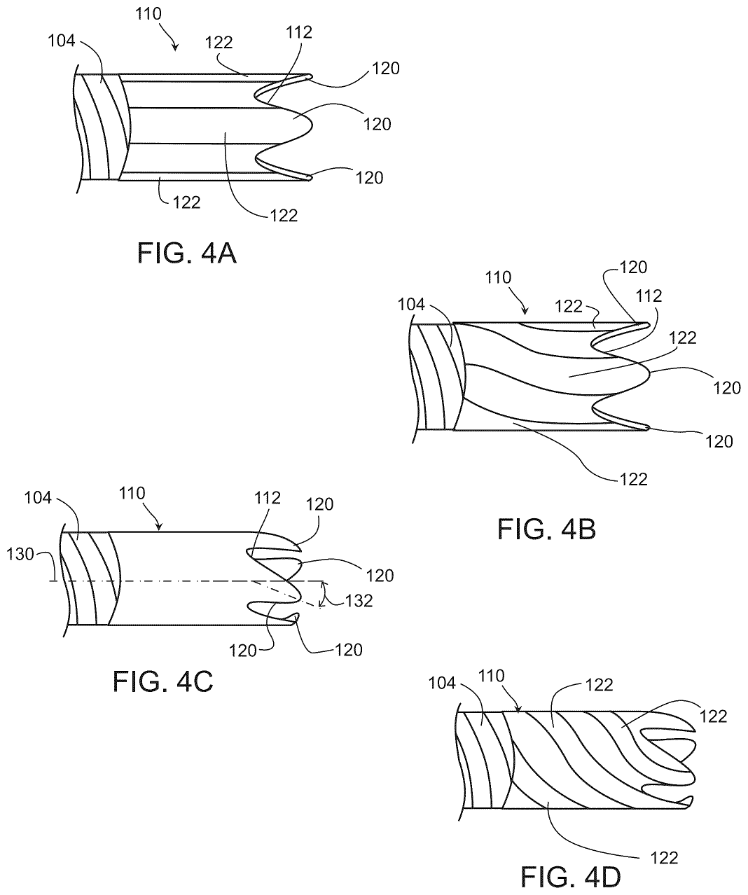

FIGS. 4A to 4D illustrate a non-exhaustive number of variations of improved navigational tips 110 for use with catheters and similar medical devices. FIG. 4A and illustrate a number of scalloped members 120 at the far end 112 of the tip structure 110 that is opposite to a catheter body 104. As shown, this variation includes a far end 112 that is sinusoidal about a perimeter of the far end 112. In addition, the tip member 110 can include any number of reinforcement regions 122, which allow the scalloped members 120 to return to a pre-determined shape after deformation, as discussed above. FIG. 4A illustrates the reinforcement member as being in alignment or extending parallel with an axis of the tip member 110. Alternatively, FIG. 4B shows the reinforced region 122 as extending helically or spirally about the tip member 110. Variations of the improved navigational tip 110 are usually fabricated from a flexible polymer with any number of reinforcement structures to stiffen regions of the tip (as shown in FIGS. 4A and 4B). However, additional variations of tip 110 can include tips fabricated from a relatively stiffer or higher durometer material, where regions 122 comprise regions of increased flexibility/lower durometer. In further variations, the arrangement of materials can cause preferential bending either outward (away from an axis of the tip structure) or inward such that the scalloped member is biased to deform towards an axis of the tip structure 110. For example, in one example, a reinforcement material can be positioned on an exterior of the scalloped member with a more flexible material on an interior such that the scalloped member bends towards the flexible material.

FIGS. 4C and 4D illustrate additional variations of tip structures 110 where the scalloped structures 120 are twisted or extend at an angle 132 relative to an axis 130 of the tip member 110. In contrast, the tip members 110 in FIGS. 4A and 4B include scalloped members 120 that extend in alignment with an axis of the tip member 110. FIG. 4D illustrates a tip member similar to FIG. 4C with the addition of regions 122 of differing durometer relative to the remainder of the tip member 110.

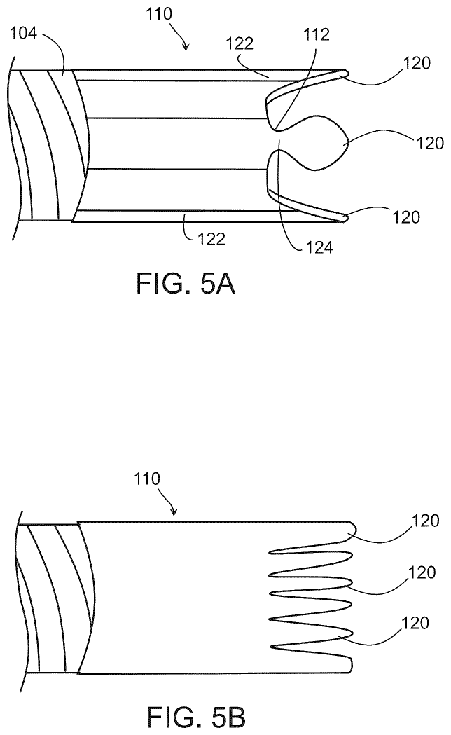

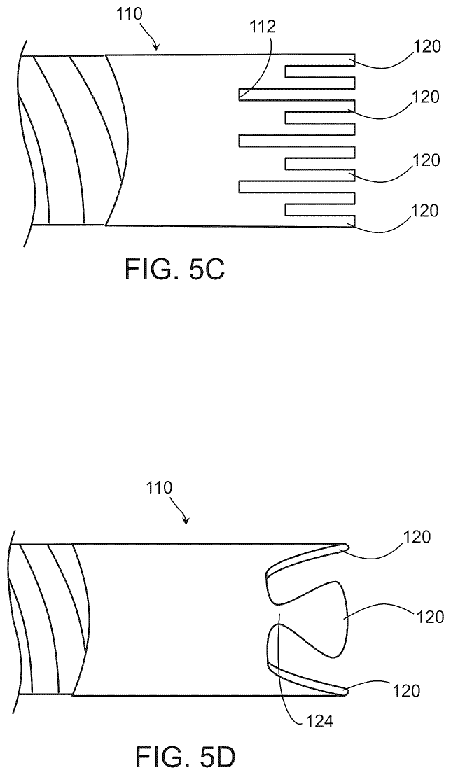

FIGS. 5A to 5D show additional variations of improved navigational tips 110. FIG. 5A illustrates a far end 112 of the tip member 110 having an irregular shape that permits a reduced area region 124 of a scalloped member 120. As with other variations, this variation of the tip member 110 can optionally include regions of varying durometer/support 122 relative to the remainder of the tip member 110. FIG. 5B illustrates a tip member 110 with scalloped members 120 of varying sizes. Alternatively, or in combination, the variation of FIG. 5C illustrates a plurality of scalloped members 120 that are formed from a specific pattern that creates scalloped members 120 of varying depth. FIG. 5D shows a variation of a tip member 110 where a scalloped member 120 increases in width from a neck 124 towards a distal end of the scalloped member 120. Additional variations of the scalloped members 120 can include scalloped members having a wall thickness that increases, decreases, or is irregular across a length of the scalloped member 120.

It is noted that the variations disclosed herein provide for a plurality of scalloped members at a far end 112 of a tip member 110. Alternate variations of the device can include scalloped members formed from an end of the catheter body 104.

As for other details of the present invention, materials and manufacturing techniques may be employed within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described, and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

We claim:

1. A catheter construction for use in a vessel, the catheter construction comprising:
a catheter body having a catheter lumen extending therethrough; and
a tip structure located at a distal end of the catheter body, where an end of the tip structure located opposite to the catheter body comprises an undulating shape forming a plurality of scalloped members, where each scalloped member of the plurality of scalloped members is configured to flex individually from an adjacent scalloped member, such that when advanced against a vessel wall, the plurality of scalloped members are configured to flex and bend independently to assist in articulation of the tip structure.

2. The catheter construction of claim 1, further comprising at least one material strip extending in the tip structure, where the at least one material strip comprises a first durometer, where the first durometer is different than a second durometer of a remainder of the tip structure.

3. The catheter construction of claim 2, wherein the first durometer is higher than the second durometer.

4. The catheter construction of claim 2, wherein the at least one material strip extends in alignment with an axis of the tip structure.

5. The catheter construction of claim 2, wherein the at least one material strip extends spirally about the tip structure.

6. The catheter construction of claim 1, wherein the plurality of scalloped members extend parallel to an axis of the tip structure.

7. The catheter construction of claim 1, wherein the plurality of scalloped members extend at an angle to an axis of the tip structure.

8. The catheter construction of claim 1, wherein at least one of the plurality of scalloped members is configured to deflect towards an axis of the tip structure.

9. The catheter construction of claim 1, wherein at least one of the plurality of scalloped members is configured to deflect outward away from an axis of the tip structure.

10. The catheter construction of claim 1, wherein at least one scalloped member of the plurality of scalloped members comprises a neck area comprising a reduced surface area relative to a remainder of the at least one scalloped member.

11. The catheter construction of claim 1, wherein at least two of the plurality of scalloped members each comprise a different shape.

12. The catheter construction of claim 1, wherein the plurality of scalloped members each comprise a similar shape.

13. The catheter construction of claim 1, wherein at least one of the plurality of scalloped members comprises a shape that narrows toward a distal tip.

14. The catheter construction of claim 1, wherein at least one of the plurality of scalloped members comprises a shape that increases toward a distal tip.

15. A catheter construction for use in a vessel, the catheter construction comprising:
a catheter body having a catheter lumen extending therethrough; and
a plurality of scalloped members located a distal end of the catheter body and formed from an undulating shape pattern such that each scalloped member of the plurality of scalloped members is configured to flex individually from an adjacent scalloped member, such that when advanced against a vessel wall, flexing of one or more of the plurality of scalloped members causes asymmetric bending of a tip of the catheter body.

16. The catheter construction of claim 1, further comprising at least one material strip extending in the tip structure, where the at least one material strip comprises a first durometer, where the first durometer is different than a second durometer of the a tip of the catheter body.

17. The catheter construction of claim 2, wherein the at least one material strip extends in alignment with an axis of the tip structure.

18. The catheter construction of claim 2, wherein the at least one material strip extends spirally about the tip structure.

19. The catheter construction of claim 1, wherein the plurality of scalloped members extend parallel to an axis of the tip structure.

20. A catheter construction for use in a vessel, the catheter construction comprising:
a catheter body having a catheter lumen extending therethrough; and
a tip structure located at a distal end of the catheter body, where an end of the tip structure located opposite to the catheter body comprises an undulating shape forming at least one scalloped member configured to flex individually from an portion of the tip structure, such that when advanced against a vessel wall, the at least one scalloped members is configured to flex and bend independently to assist in articulation of the tip structure.
